(19) United States
(12) Patent Application Publication
(10) Pub. No.: US 2025/0006371 A1
(43) Pub. Date: Jan. 2, 2025

(54) EARLY WARNING SYSTEM FOR HYPERTENSION PATIENTS

(71) Applicant: BAYER AKTIENGESELLSCHAFT, Leverkusen (DE)

(72) Inventors: Hans-Peter PODHAISKY, Leverkusen (DE); Toeresin KARAKOYUN, Leverkusen (DE); Daniel PAULSON, Whippany, NJ (US); Daniel Franz FREITAG, Leverkusen (DE); Frank KRAMER, Leverkusen (DE); Michael KREMLIOVSKY, Whippany, NJ (US); Ying CHEN, Leverkusen (DE); Eren Metin ELCI, Leverkusen (DE)

(21) Appl. No.: 18/710,288

(22) PCT Filed: Nov. 14, 2022
(86) PCT No.: PCT/EP2022/081711

§ 371 Date: May 15, 2024

Related U.S. Application Data
(60) Provisional application No. 63/280,377, filed on Nov. 17, 2021.

(51) Int. Cl. G16H 50/20; A61B 5/00; A61B 5/021; G16H 10/60; G16H 50/30

(52) U.S. Cl. CPC G16H 50/20; A61B 5/021; A61B 5/746; G16H 10/60; G16H 50/30

(57) ABSTRACT

Systems, methods, and computer programs disclosed herein relate to the recognition of threatening conditions in patients suffering from hypertension.

### FIELD

Systems, methods, and computer programs disclosed herein relate to the recognition of threatening conditions in patients suffering from hypertension.

### BACKGROUND

Hypertension, also known as high or raised blood pressure, is a condition in which the blood vessels have persistently raised pressure. Blood is carried from the heart to all parts of the body in the vessels. Each time the heart beats, it pumps blood into the vessels. Blood pressure is created by the force of blood pushing against the walls of blood vessels as it is pumped by the heart. The higher the pressure, the harder the heart has to pump.

Hypertension is a serious medical condition and can increase the risk of heart, brain, kidney and other diseases. It is a major cause of premature death worldwide. Hypertensive crisis is an umbrella term for hypertensive urgency and hypertensive emergency. These two conditions occur when blood pressure becomes very high, possibly causing organ damage. Hypertensive urgency is a marked elevation in blood pressure without evidence of target organ damage, such as pulmonary edema, cardiac ischemia, neurologic deficits, or acute renal failure. A hypertensive emergency is very high blood pressure with potentially life-threatening symptoms and signs of acute damage to one or more organ systems (especially brain, eyes, heart, aorta, or kidneys). It is different from a hypertensive urgency by this additional evidence for impending irreversible hypertension-mediated organ damage.

There is a need for solutions to identify a hypertensive crisis in an individual as early as possible and/or to take the actions necessary to prevent and/or reduce harm to the individual when a hypertensive crisis occurs.

### SUMMARY

The present invention provides means to detect hypertensive crisis in a person at an early stage so that the person or another person, such as a physician or nurse, can take action to improve the person's condition.

In a first aspect, the present disclosure provides a computer-implemented method, the method comprising:
receiving patient data, the patient data comprising data about a patient's blood pressure over time,
analyzing the patient data and identifying one or more signs of a hypertensive crisis in the patient data,
providing a notification to the patient, the notification comprising information about the identified signs of the hypertensive crisis and a request for the patient to perform an action.

In another aspect, the present disclosure provides a computer system comprising:
a processor; and
a memory storing an application program configured to perform, when executed by the processor, an operation, the operation comprising:
   receiving patient data, the patient data comprising data about a patient's blood pressure over time,
   analyzing the patient data and identifying one or more signs of a hypertensive crisis in the patient data,
   providing a notification to the patient, the notification comprising information about the identified signs of the hypertensive crisis and a request for the patient to perform an action.

In another aspect, the present disclosure provides a non-transitory computer readable medium having stored thereon software instructions that, when executed by a processor of a computer system, cause the computer system to execute the following steps:
receiving patient data, the patient data comprising data about a patient's blood pressure over time,
analyzing the patient data and identifying one or more signs of a hypertensive crisis in the patient data,
providing a notification to the patient, the notification comprising information about the identified signs of the hypertensive crisis and a request for the patient to perform an action.

Further aspects as well as preferred embodiments can be found in the patent claims, the present description and in the drawings.

### DETAILED DESCRIPTION

The invention will be more particularly elucidated below without distinguishing between the aspects of the invention (method, computer system, computer-readable storage medium). On the contrary, the following elucidations are intended to apply analogously to all the aspects of the invention, irrespective of in which context (method, computer system, computer-readable storage medium) they occur.

If steps are stated in an order in the present description or in the claims, this does not necessarily mean that the invention is restricted to the stated order. On the contrary, it is conceivable that the steps can also be executed in a different order or else in parallel to one another, unless one step builds upon another step, this absolutely requiring that the building step be executed subsequently (this being, however, clear in the individual case). The stated orders are thus preferred embodiments of the invention.

As used herein, the articles “a” and “an” are intended to include one or more items and may be used interchangeably with “one or more” and “at least one.” As used in the specification and the claims, the singular form of “a”, “an”, and “the” include plural referents, unless the context clearly dictates otherwise. Where only one item is intended, the term “one” or similar language is used. Also, as used herein, the terms “has”, “have”, “having”, or the like are intended to be open-ended terms. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

Some implementations will be described more fully hereinafter with reference to the accompanying drawings, in which some, but not all implementations are shown. Indeed, various implementations may be embodied in many different forms and should not be construed as limited to the implementations set forth herein; rather, these example implementations are provided so that this disclosure will be thorough and complete, and will fully convey the scope of the disclosure to those skilled in the art.

The present disclosure provides means for recognition of threatening conditions in patients suffering from hypertension.

The threatening conditions can be a hypertensive urgency and/or hypertensive emergency, collectively referred to herein as hypertensive crisis. This means that the term hypertensive crisis in the present description may have the following meanings: hypertensive urgency, or hypertensive emergency, or hypertensive urgency and hypertensive emergency.

The detection of a threatening condition is based on patient data. Patient data can provide information about the patient's general and/or current anatomical, physiological and/or physical and/or behavioral condition.

The patient data comprise data about the patient's blood pressure over time.

“Blood pressure” is the force of circulating blood on blood vessels per unit area. Most of this pressure results from the heart pumping blood through the circulatory system. Blood pressure varies along the blood circulation and is highest in the aorta and continues to drop as blood travels through the circulatory system via arteries, capillaries, and veins. When used without qualification, the term “blood pressure” usually refers to the pressure in the large arteries (arterial blood pressure).

In addition, (arterial) blood pressure varies continuously over the cardiac cycle, but in clinical practice only systolic and diastolic pressures are routinely reported. The systolic pressure is the maximum pressure during one heartbeat and the diastolic pressure is the minimum pressure between two heartbeats in the cardiac cycle. Systolic and diastolic pressures are usually expressed in millimeters of mercury (mmHg) above ambient atmospheric pressure.

For decades, systolic and diastolic pressures were measured almost exclusively in the brachial artery with blood pressure cuff (peripheral blood pressure). However, the shape of the pressure waveform changes continuously throughout the arterial tree. Although diastolic and mean arterial pressures are relatively constant, systolic pressure may be up to 40 mmHg higher in the brachial artery than in the aorta. This phenomenon of systolic pressure amplification arises principally because of an increase in arterial stiffness moving away from the heart: as the pressure wave travels from the highly elastic central arteries to the stiffer brachial artery, the upper portion of the wave becomes narrower, the systolic peak becomes more prominent, and systolic pressure increases.

Central blood pressure is the pressure in the aorta, which is the large artery into which the heart pumps and is considered a better indicator of the pressure the heart and other vital organs experience than the peripheral blood pressure. Furthermore, central blood pressure has been shown to be a better predictor of vascular disease when compared to peripheral blood pressure.

Mean arterial pressure is the average arterial pressure throughout one cardiac cycle, systole, and diastole.

It should be noted that the present invention is not limited to any particular definition or expression of blood pressure, nor is it limited to any particular method of measurement. It is only important that the measured blood pressure values allow a statement to be made about the patient's state of health and that the measured blood pressure values indicate the occurrence and/or presence of a hypertensive crisis.

Thus, when the present disclosure refers to blood pressure, it may mean (arterial) systolic and/or diastolic arterial blood pressure, peripheral blood pressure, central blood pressure, mean (arterial) blood pressure, blood pressure values over full cardiac cycles, and/or derivatives of the foregoing, e.g., integration of pressure over time, and/or others.

In addition, the term blood pressure should not be understood to mean that the measurand recorded is actually a pressure. The measurand may also be another parameter that correlates with blood pressure. For example, there are also optical methods for measuring blood pressure (see for example L. A. Geddes: Handbook of Blood Pressure Measurement, Springer 2014, ISBN 9781468471717).

The patient's blood pressure is measured continuously using one or more sensors.

The term “continuous(ly)”, as it is used herein, means that blood pressure measurements are taken at defined intervals over a period of at least one day, even more preferably at least one week, even more preferably at least one month, most preferably about a period of more than one month. The term “continuous(ly)” does not necessarily mean that one measurement is taken immediately after the other.

Instead, the blood pressure values are measured at defined time intervals, with these intervals being no greater than one day, preferably no greater than one hour.

Preferably, blood pressure is measured automatically, i.e., without the patient's intervention.

Blood pressure measurements can be regular or irregular, the intervals between two consecutive measurements can be constant or variable. Preferably, the intervals between two consecutive measurements are not greater than one hour, preferably not greater than 15 minutes, even more preferably not greater than 10 minutes.

In a preferred embodiment, the rate at which the blood pressure is measured is greater than twice the maximum frequency of the blood pressure variations over time (in accordance with the Nyquist-Shannon sampling theorem). However, the term “rate” should not be understood to mean that the interval between two measurement points is constant. It is irrelevant whether the blood pressure is measured at constant or variable intervals. If the blood pressure is measured at variable intervals, the “sampling rate” preferably represents the largest distance between two consecutive measuring points.

Preferably, the rate at which blood pressure is measured, is specified individually for each patient. Preferably, the rate is determined (at least partially) based on patient's blood pressure variation over time. If a patient experiences greater variation, a higher rate may be set for that patient than for a patient who experiences less variation.

The rate can also change over the course of a day. For example, it is possible to measure the blood pressure less frequently during periods of time in which experience has shown that only minor fluctuations in blood pressure occur for many patients or for the patient under consideration than during periods of time in which the fluctuations are greater and/or in which greater deviations occur.

Preferably, the rate at which blood pressure measurements are taken is adjusted overtime to the degree of change of blood pressure. Preferably, the sensor (or a controller controlling the sensor) is configured to shorten the intervals between two successive measurements when the blood pressure increases and/or when the blood pressure increases more rapidly than a pre-defined threshold, and/or when the blood pressure exceeds a pre-defined threshold. If the blood pressure drops and/or the blood pressure falls below a defined value, the intervals between two successive measurements can be extended again.

Blood pressure can be measured with one or more sensors, which are preferably placed in or on the patient's body. In addition, contactless sensors can be used to measure or estimate blood pressure (see, e.g.: N. Sugita et al.: “Contactless Technique for Measuring Blood-Pressure Variability from One Region in Video Plethysmography”, Journal of medical and Biological Engineering, 2019, 39, 76-85).

Examples of sensors worn by the patient on patient's body that continuously measure blood pressure can be found, e.g., in U.S. Ser. No. 10/568,527B2, EP3609394A1, U.S. Ser. No. 10/952,623B2, and WO2019/172569A1.

Examples of sensors that continuously measure blood pressure in the patient's body can be found, e.g., in WO2006/023786A2, EP2397185B1, U.S. Pat. No. 8,602,999B2, and WO2004/014456A2.

As described above, for blood pressure measurement all current or new methods can be considered. In other words: the present invention is not limited to any particular method of determining blood pressure or to any particular device or type of device for blood pressure measurement. It is also possible to use different methods and/or devices and/or types of devices.

The patient data (including the blood pressure data) are analyzed in order to identify one or more signs of a hypertensive crisis in the patient data.

A sign of a hypertensive crisis may be, for example, a value of the measured blood pressure that is above a pre-defined threshold. So, in one embodiment of the present disclosure, a sign of the presence of a hypertensive crisis is a blood pressure greater than a pre-defined threshold. The blood pressure can be the last measured value, or it can be a mean value (e.g., the arithmetic mean) of the last n measured values, where n is an integer greater than 1 (e.g., 2 or 3 or 4 or 5 or any other number). The blood pressure can also be a predicted blood pressure (see below for more details).

Specific thresholds for the presence of hypertensive urgency and hypertensive emergency have been proposed. According to the publication of R. Gauer et al., hypertensive urgency is characterized by a systolic blood pressure of 180 mmHg or more, or a diastolic blood pressure of 110 mmHg or more (Severe Asymptomatic Hypertension: Evaluation and Treatment, Am Fam Physician. 2017 Apr. 15; 95(8): 492-500). According to the publication of C. K. Vaidya and J. R. Ouellette, hypertensive emergency is characterized by a blood pressure higher than 180/120 mmHg (Hypertensive Urgency and Emergency, Hypertensive Urgency and Emergency, March 2007, pages 43-50).

So, preferred pre-defined thresholds are 180/110 mmHg and/or 180/120 mmHg.

However, other values have also been proposed and, finally, all these values are arbitrarily derived numbers that have not been associated with short-term morbidity or mortality (W. D. Alley et al.: Hypertensive Urgency, StatPearls Publishing, 2021, https://www.ncbi.nlm.nih.gov/books/NBK513351/).

Therefore, in a preferred embodiment of the present disclosure, the threshold is determined (pre-defined) by a physician. Preferably an individual threshold is determined (pre-defined) for each patient.

In another preferred embodiment of the present disclosure, the threshold is determined automatically on the basis of patient data. For example, it is conceivable that a lower threshold is set for patients who have already experienced a hypertensive crisis than for patients who have not yet experienced a hypertensive crisis. Additionally, or alternatively, the threshold may be set lower for patients who have been diagnosed by a physician as being at higher risk of permanent health damage from the occurrence of a hypertensive crisis.

An expert system or a machine learning model trained on data from a large number of patients can use patient data to automate setting a threshold or recommending a threshold.

Patient data may include other (further) patient data in addition to blood pressure data.

Further patient data can be provided by the patient or any other person such as a physician and/or a nurse.

Further patient data can be captured (e.g., automatically) by one or more sensors (e.g., motion sensor, activity tracker, blood glucose meter, heart rate meter, thermometer, impedance sensor, microphone (e.g., for voice analysis) and/or others).

Further patient data can include, e.g.: age, gender, body size, body weight, body mass index, resting heart rate, heart rate variability, sugar concentration in urine, body temperature, impedance, lifestyle information about the life of the patient, such as consumption of alcohol, smoking, and/or exercise and/or the patient's diet, medical intervention parameters such as regular medication, occasional medication, or other previous or current medical interventions and/or other information about the patient's previous and current treatments and reported health conditions and/or combinations thereof.

Further patient data can comprise information from an EMR (electronic medical record, also referred to as EHR (electronic health record)). The EMR may contain information about a hospital's or physician's practice where certain treatments were performed and/or certain tests were performed, as well as various other meta-information about the patient's treatments, medications, tests, and physical and/or mental health records.

Further patient data can comprise information about a person's condition obtained from the person himself/herself (self-assessment data, (electronic) patient reported outcome data (e)PRO)). Besides objectively acquired anatomical, physiological and/or physical data, the well-being of the patient also plays an important role in the monitoring of health. Subjective feeling can also make a considerable contribution to the understanding of objectively acquired data and of the correlation between various data. If, for example, it is captured by sensors that a person has experienced a physical strain, for example because the respiratory rate and the heart rate have risen, this may be because just low levels of physical exertion in everyday life place a strain on the person; however, another possibility is that the person consciously and gladly brought about the situation of physical strain, for example as part of a sporting activity. A self-assessment can provide clarity here about the causes of physiological features.

Subjective feeling can be collected by use of a self-assessment unit, with the aid of which the patient can record information about subjective health status. Preference is given to a list of questions which are to be answered by a patient. Preferably, the questions are answered with the aid of a computer system, e.g., the computer system of the present disclosure (which may be a desktop computer, a laptop computer a tablet computer and/or a smartphone). One possibility is that the patient has questions displayed on a screen and/or read out via a speaker. One possibility is that the patient inputs information into a computer system by, e.g., inputting text via an input device (e.g., keyboard, mouse, touchscreen and/or a microphone (by means of speech input)). It is conceivable that a chatbot is used in order to facilitate the input of all items of information for the patient. It is conceivable that the questions are recurring questions which are to be answered once or more than once a day or a week by a patient. It is conceivable that some of the questions are asked in response to a defined event. It is, for example, conceivable that it is captured by means of one or more sensors that a physiological parameter is outside a defined range (e.g., an increased respiratory rate is established and/or the blood pressure exceeds a pre-defined threshold). As a response to this event, the patient can, for example, receive a message via his/her smartphone or smartwatch or the like that a defined event has occurred and that said patient should please answer one or more questions, for example in order to find out the causes and/or the accompanying circumstances in relation to the event.

In another preferred embodiment, a trained machine learning model is used to analyze patient data and identify one or more signs of a hypertensive crisis in the patient data.

Such a “machine learning model”, as used herein, may be understood as a computer implemented data processing architecture. The machine learning model can receive input data and provide output data based on that input data and the machine learning model, in particular parameters of the machine learning model. The machine learning model can learn a relation between input data and output data through training. In training, parameters of the machine learning model may be adjusted in order to provide a desired output for a given input.

The input data can be patient data. The output data can be, e.g., a probability value, the probability value indicating the probability of (e.g., imminent) occurrence of a hypertensive crisis. The output data can also be a predicted blood pressure, i.e., a blood pressure expected for the immediate future (e.g., the expected blood pressure in 10 minutes and/or in 15 minutes and/or in 20 minutes and/or in 30 minutes and/or in 40 minutes and/or in 45 minutes and/or in 50 minutes and/or in an hour and/or in 1½ hours and/or at some other time and/or time period in the future).

The process of training a machine learning model involves providing a machine learning algorithm (that is the learning algorithm) with training data to learn from. The term “trained machine learning model” refers to the model artifact that is created by the training process. The training data must contain the correct answer, which is referred to as the target. The learning algorithm finds patterns in the training data that map input data to the target, and it outputs a trained machine learning model that captures these patterns.

The training data can comprise, for each reference patient of a multitude of reference patients, data about the occurrence and non-occurrence of one or more hypertensive crises (output data/target data) and the conditions under which they have occurred or not occurred (input data/patient data).

The term “multitude” as it is used herein means an integer greater than 1, usually greater than 10, preferably greater than 100.

In the training process, training data are inputted into the machine learning model and the machine learning model generates an output. The output is compared with the (known) target. Parameters of the machine learning model are modified in order to reduce the deviations between the output and the (known) target to a (defined) minimum.

In general, a loss function can be used for training to evaluate the machine learning model. For example, a loss function can include a metric of comparison of the output and the target. The loss function may be chosen in such a way that it rewards a wanted relation between output and target and/or penalizes an unwanted relation between an output and a target. Such a relation can be, e.g., a similarity, or a dissimilarity, or another relation.

A loss function can be used to calculate a loss for a given pair of output and target. The aim of the training process can be to modify (adjust) parameters of the machine learning model in order to reduce the loss to a (defined) minimum.

If, for example, the output and the target are numbers (such as probability values for the occurrence of a hypertensive crisis or predicted blood pressure value(s)), the loss can be the absolute value of the difference of the numbers. In this case, a high absolute value of the loss function can mean that a parameter of the model needs to undergo a strong change.

Once the machine learning model is trained, it can be used to detect an (e.g., imminent) hypertensive crisis on the basis of patient data inputted into the machine learning model.

In the event that indications (signs) of the occurrence or presence of a hypertensive crisis are identified based on the patient data, a notification is transmitted and/or outputted to the patient. Such a notification to the patient in response to the identification of signs for occurrence or presence of a hypertensive crisis is also referred to herein as “first notification”.

The first notification indicates to the patient that a hypertensive crisis is imminent or already present (as the case may be).

The first notification can be or comprise an acoustic, optical and/or tactile signal to attract the patient's attention. For example, it is conceivable that a mobile device (e.g., cell phone or smartwatch) of the patient is caused to generate one or more vibrations, to generate one or more sounds, to play one or more audio recordings, and/or to display one or more visually perceptible messages in the form of text, graphics, and/or images on a display.

The first notification can contain information about the impending or existing hypertensive crisis. The first notification can contain information about what the patient can do and/or should do to improve the health condition and/or prevent harm.

The first notification comprises a request for the patient to perform an action.

There may be two reasons to request an action be taken by the patient.

First, requesting an action to be taken can verify that the patient has received and understood the notification. In other words: the aim of the request can be to ensure that the patient knows that a hypertensive crisis is imminent or already present (as the case may be). If the patient does not react, i.e., does not perform the requested action, this is an indication that the patient has not received the notification and/or has not grasped it and/or is unable to perform the action (for example, because the patient is confused or unconscious due to the elevated blood pressure).

Second, the request to perform an action can serve to initiate measures that should lead to the patient's health condition not deteriorating further and/or that should lead to the patient's health condition improving.

The requested action may be, for example, the pressing of a real or a virtual switch on a computer, the wiping away of a graphic on a touch-sensitive display, the issuance of a command by voice, and/or any other action that shows that the patient has received, grasped, and understood the communication.

One or more reminders can be transmitted and/or outputted to the patient if the patient has not performed the required action within a pre-defined period of time (e.g., within one or more minutes).

The first notification can include a request for the patient to answer one or more questions and/or provide one or more information to confirm the finding (hypertensive crisis) and/or to obtain further information about the severity and/or potential risks and/or to initiate action to treat the patient.

Questions can be answered, or information can be provided, for example, by the patient clicking on the appropriate answer or clicking on the appropriate information e.g., displayed on a screen.

Preferably, questions are asked and/or prompts are designed in a way that allows the patient to select one option from a number of options, with the number of options kept to a minimum (e.g., two, three, or four options).

It is also conceivable that the patient is given a number of symptoms and is asked to select all those symptoms that apply to him/her (e.g., in the current situation).

It is also conceivable that the patient is asked to provide one or more statements verbally. With the help of a microphone, the patient's information can be recorded. It is conceivable that the patient's voice is analyzed in order to confirm the finding (hypertensive crisis), determine the severity, assess the risk to the patient, and/or obtain further information about the patient's condition based on the analysis.

The patient may provide one or more of the following information: presence or severity of the following symptoms: chest pain, headache, stomach pain, nausea, vomiting, back pain, breathing difficulty, vision problems (including sudden blindness), decreased urination, arm and/or leg weakness, confusion, seizures, loss of consciousness, mood and/or personality changes.

The first notification may comprise instructions to the patient that the patient should follow to improve the health condition, i.e., to prevent and/or overcome the hypertensive crisis.

Measures to be taken by the patient may be: remaining calm, lying down, taking one or more medications, calling the emergency physician. Preferably, the patient is asked to confirm actions (e.g., by pressing a real or virtual switch on a computer system, by wiping away a graphic on a touch-sensitive display, by the issuance of a command by voice, and/or any other action that can be interpreted by a computer system as a confirmation).

It is also conceivable that the patient wears (in the body or on the body) a device for automated dispensing of one or more medications. It is conceivable that in the event of signs of the emergence or presence of a hypertensive crisis, the device is caused to administer one or more medications to lower blood pressure and/or to prevent and/or reduce damage from the excessive blood pressure. Medication dispensing devices are described in prior art (see, e.g., WO2015/097323A1, WO2002/76533A1, US20070112326A1).

In a preferred embodiment, the first notification and/or any subsequent notification to the patient (such as a reminder) includes a means to contact/notify a physician or emergency physician. Preferably, the notification comprises a virtual button, the actuation of which informs a physician or emergency physician of the patient's condition (and preferably the location of the patient). It is also conceivable that a physician or emergency physician is automatically contacted if the patient is unable to form clear words and/or gives a defined voice command (e.g.: “help”).

As already described above, preferably the rate of blood pressure measurements is increased (e.g., to a rate of every minute or even more frequently) as long as the blood pressure remains above a pre-defined threshold (e.g., above 180/110 mmHg or 180/120 mmHg).

It is also conceivable that, as an alternative to the patient or in addition to the patient, another person is informed about an impending and/or existing hypertensive crisis. Such other person can, for example, be a physician, a nurse, a relative and/or a close friend.

A notification transmitted to someone other than the patient to inform of an impending or present hypertensive crisis in the patient is also referred to herein as “second notification”.

The second notification can be transmitted to the other person, for example, via e-mail, SMS message, automated phone call and/or the like. The second notification preferably includes information about the patient, the patient's location, and/or the patient's medical condition. Preferably, the geo-coordinates of a computer system typically used and/or carried by the patient (such as a mobile phone or smartwatch) are acquired and transmitted. Preferably, a communication link is automatically established between the patient and the other person so that the other person can have a calming effect on the patient and/or monitor the patient's condition and/or give instructions to the patient.

Preferably, the information transmitted to the other person includes the patient's current blood pressure data as well as the time history of blood pressure values during a defined period of time prior to the current time (e.g., the last hour or couple of hours). Preferably, the other person can display the entire recorded history and determine which period should be displayed.

Preferably, each newly measured blood pressure value and optionally other measured values concerning the patient's physical condition are transmitted to the other person for a defined period of time and displayed to the other person in order to allow the other person to monitor the patient's health condition.

The second notification may be sent to the other person in case the patient does not perform the requested action within a defined period of time and/or in case the patient's blood pressure does note decrease below a predefined first threshold and/or the patient's blood pressure continues to increase and/or the patient's blood pressure increases above a predefined second threshold.

Thus, such a second notification can be a further escalation stage intended to ensure that the patient's health does not deteriorate further. It is conceivable that further levels of escalation are present. For example, a second notification can be sent first to a patient's relative or to a caring physician or to a nurse. Another second message can be sent to an emergency physician ifthe patient's condition does not improve within a defined period of time or if it deteriorates.

The invention is explained in more detail below with reference to the drawings, without limiting the invention to the features and combinations of features shown in the drawings.

FIG. 1 shows schematically by way of example one embodiment of the computer system according to the present disclosure.

Generally, a computer system of exemplary implementations of the present disclosure may be referred to as a computer and may comprise, include, or be embodied in one or more fixed or portable electronic devices.

The computer system (1) comprises an input unit (2), a processing unit (3), a memory (4), an output unit (5), and communication interface(s) (6).

Data (e.g., patient data) can be entered into the computer system (1) via the input unit (2). The input unit (2) can act as an interface to a user by accepting commands to control the computer system (1). User input interface(s) may be wired or wireless, and may be configured to receive information from a user into the computer system (1), such as for processing, storage and/or display. Suitable examples of user input interfaces include a microphone, image or video capture device (e.g., a camera), keyboard or keypad, joystick, touch-sensitive surface (separate from or integrated into atouchscreen) and/or the like.

In some examples, the user interfaces may include automatic identification and data capture (AIDC) technology for machine-readable information. This may include barcode, radio frequency identification (RFID), magnetic stripes, optical character recognition (OCR), integrated circuit card (ICC), and the like. The user interfaces may further include one or more interfaces for communicating with peripherals such as sensors, printers, and/or the like.

The processing unit (3) may be composed of one or more processors alone or in combination with one or more memories. The processing unit (3) is generally any piece of computer hardware that is capable of processing information such as, for example, data, computer programs and/or other suitable electronic information. The processing unit (3) is composed of a collection of electronic circuits some of which may be packaged as an integrated circuit or multiple interconnected integrated circuits (an integrated circuit at times more commonly referred to as a “chip”). The processing unit (3) may be configured to execute computer programs, which may be stored onboard the processing unit (3) or otherwise stored in the memory (4). The processing unit (3) may be a number of processors, a multi-core processor or some other type of processor, depending on the particular implementation. For example, it may be a central processing unit (CPU), a field programmable gate array (FPGA), a graphics processing unit (GPU) and/or a tensor processing unit (TPU). Further, the processing unit (3) may be implemented using a number of heterogeneous processor systems in which a main processor is present with one or more secondary processors on a single chip. As another illustrative example, the processing unit (3) may be a symmetric multi-processor system containing multiple processors of the same type. In yet another example, the processing unit (3) may be embodied as or otherwise include one or more ASICs, FPGAs or the like. Thus, although the processing unit (3) may be capable of executing a computer program to perform one or more functions, the processing unit (3) of various examples may be capable of performing one or more functions without the aid of a computer program. In either instance, the processing unit (3) may be appropriately programmed to perform functions or operations according to example implementations of the present disclosure.

The memory (4) is generally any piece of computer hardware that is capable of storing information such as, for example, data, computer programs (computer-readable program code) and/or other suitable information either on a temporary basis and/or a permanent basis. The memory (4) may include volatile and/or non-volatile memory and may be fixed or removable. Examples of suitable memory include random access memory (RAM), read-only memory (ROM), a hard drive, a flash memory, a thumb drive, a removable computer diskette, an optical disk, a magnetic tape, or some combination of the above. Optical disks may include compact disk-read only memory (CD-ROM), compact disk-read/write (CD-R/W), DVD, Blu-ray disk or the like. In various instances, the memory may be referred to as a computer-readable storage medium. The computer-readable storage medium is a non-transitory device capable of storing information and is distinguishable from computer-readable transmission media such as electronic transitory signals capable of carrying information from one location to another. Computer-readable medium as described herein may generally refer to a computer-readable storage medium or computer-readable transmission medium.

Information and/or data can be output from the computer system (1) via the output unit (5). The output unit (5) may be or comprise one or more component(s) that provide(s) output information from the computer system (1), e.g., a display, a speaker, one or more light-emitting diodes (LEDs), a printer, a vibration unit (e.g., for the generation of vibration alerts) and/or the like. The display may be configured to present or otherwise display information to a user, suitable examples of which include a liquid crystal display (LCD), light-emitting diode display (LED), plasma display panel (PDP) or the like.

The computer system (1) further comprises one or more interfaces for transmitting and/or receiving information from or to other devices. Such communications interface(s) (6) may be configured to transmit and/or receive information, such as to and/or from one or more sensors, other computer(s), network(s), database(s), medication dispensing devices, or the like. The communications interface(s) (6) may be configured to transmit and/or receive information by physical (wired) and/or wireless communications links. The communications interface(s) (6) may include interface(s) to connect to a network, such as using technologies such as cellular telephone, Wi-Fi, satellite, cable, digital subscriber line (DSL), fiber optics and the like. In some examples, the communications interface(s) may include one or more short-range communications interfaces configured to connect devices using short-range communications technologies such as NFC, RFID, Bluetooth, Bluetooth LE, ZigBee, infrared (e.g., IrDA) or the like.

As indicated above, program code instructions may be stored in memory (4), and executed by processing unit (3) that is thereby programmed, to implement functions of the computer system (1), subsystems, tools and their respective elements described herein. As will be appreciated, any suitable program code instructions may be loaded onto a computer system or other programmable apparatus from a computer-readable storage medium to produce a particular machine, such that the particular machine becomes a means for implementing the functions specified herein. These program code instructions may also be stored in a computer-readable storage medium that can direct a computer, processing unit or other programmable apparatus to function in a particular manner to thereby generate a particular machine or particular article of manufacture. The instructions stored in the computer-readable storage medium may produce an article of manufacture, where the article of manufacture becomes a means for implementing functions described herein. The program code instructions may be retrieved from a computer-readable storage medium and loaded into a computer, processing unit or other programmable apparatus to configure the computer, processing unit or other programmable apparatus to execute operations to be performed on or by the computer, processing unit or other programmable apparatus.

Retrieval, loading and execution of the program code instructions may be performed sequentially such that one instruction is retrieved, loaded and executed at a time. In some example implementations, retrieval, loading and/or execution may be performed in parallel such that multiple instructions are retrieved, loaded, and/or executed together. Execution of the program code instructions may produce a computer-implemented process such that the instructions executed by the computer, processing circuitry or other programmable apparatus provide operations for implementing functions described herein.

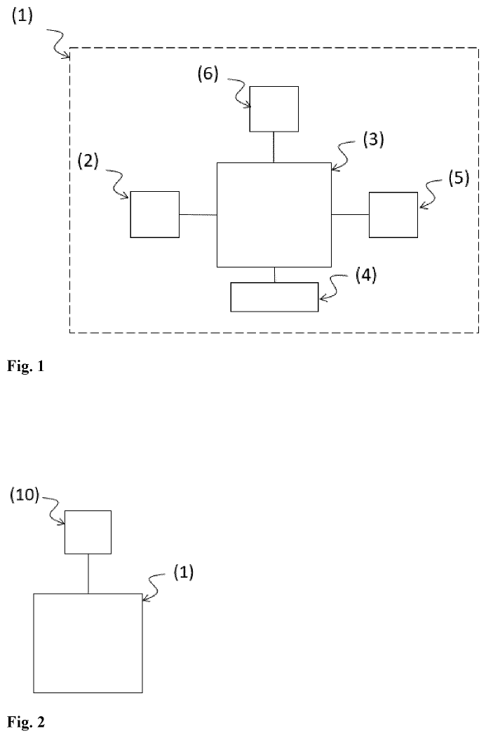

FIG. 2 shows schematically by way of example a computer system (1) according to the present disclosure connected to one or more sensors (10).

The computer system (1) may be designed as a smartwatch, a mobile phone (smartphone), a tablet computer, a desktop computer, or a computer tower. The computer system (1) is usually used by the patient.

The one or more sensors (10) can be connected to the computer system (1) via cable or via a short-range communications technology such as NFC, RFID, Bluetooth, Bluetooth LE, ZigBee, infrared and/or the like. The one or more sensors (10) can also be part of the computer system (1).

The one or more sensors (10) are configured to continuously measure the blood pressure of a patient (and optionally further patient data). The measured blood pressure data (and optionally further patient data) are transmitted to the computer system (1) and analyzed.

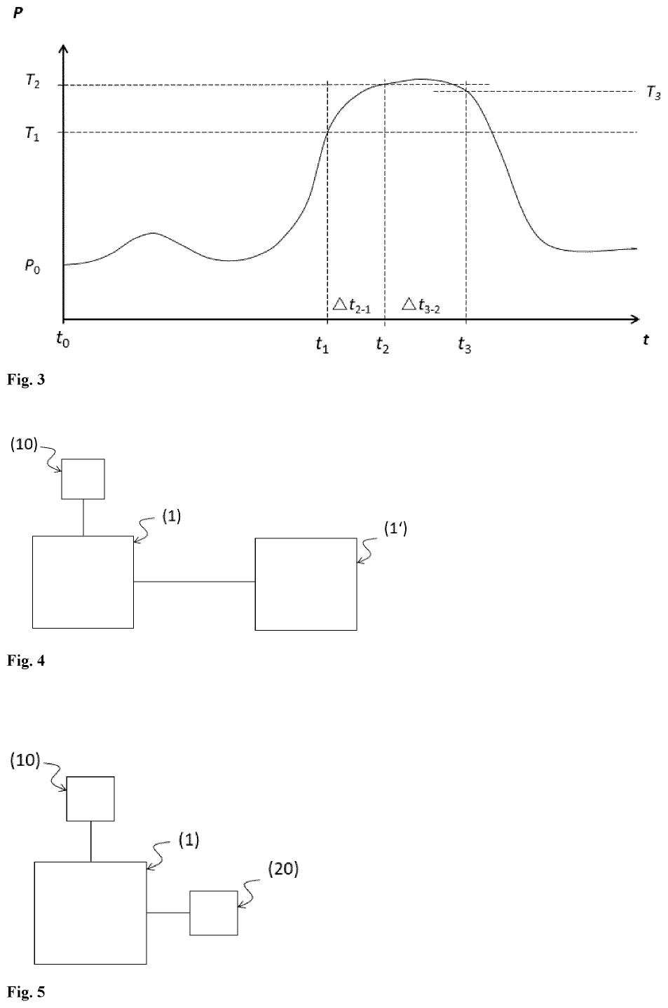

FIG. 3 shows schematically by way of example the analysis of patient data and the identification of signs for the occurrence and/or presence of a hypertensive crisis.

FIG. 3 shows the course of a parameter P as a function of time t.

The parameter P may represent the patient's (measured and/or predicted) blood pressure, such as the systolic blood pressure or diastolic blood pressure, or a combination thereof, or some other expression of blood pressure, or the parameter P may represent a probability of the occurrence or presence of a hypertensive crisis, or it may represent some other parameter that correlates with the probability of the occurrence or presence of a hypertensive crisis. It is also conceivable that several parameters are determined, and several criteria must be satisfied for the computer system to recognize signs of an emerging or existing hypertensive crisis. For example, a blood pressure value that exceeds a pre-defined first threshold, in combination with a slope of the blood pressure curve as a function of time that is greater than a pre-defined second threshold, may indicate a crisis.

At the time point to the parameter P has the value Po. The parameter P then increases over time, passes through a local maximum, followed by a minimum, and then increases sharply.

At the time point t₁, the parameter P exceeds a pre-defined threshold T₁. This can be a sign of the occurrence or presence of a hypertensive crisis. This event can trigger the provision of the first notification to the patient. The first notification comprises a request for the patient to perform an action.

The computer system can be configured in various ways:

A reminder can be provided to the patient if the patient does not perform the requested action within a defined period of time (e.g., within the time interval Δt₂₋₁).

A reminder or a further notification can be provided to the patient if the parameter P exceeds a second pre-defined threshold (e.g., the threshold T₂).

A notification can be transmitted to another person (e.g., a physician, a nurse, a relative and/or any other person with a relation to the patient) if the patient does not perform the requested action within a defined period of time (e.g., within the time interval Δt₂₋₁).

A notification can be transmitted to another person (e.g., a physician, a nurse, a relative and/or any other person with a relation to the patient) if the parameter P exceeds a second pre-defined threshold (e.g., the threshold T₂).

A notification can be transmitted to another person (e.g., a physician, a nurse, a relative and/or any other person with a relation to the patient) if the patient has performed the requested action within a pre-defined time period (within the time interval Δt₂₋₁), however, the parameter P does not fall below a pre-defined third threshold (e.g., the threshold T₃) within a pre-defined period of time (e.g., within the time interval Δt₃₋₂).

Preferably, the first computer system can be individually configured for the patient by a physician and/or another person. Such configuration may include specifying the following: the conditions under which a first notification is provided to the patient (e.g., at what blood pressure a notification is provided), the content of the first notification (e.g., what information is given to the patient, what information is requested from the patient, what actions are requested from the patient, what measures should the patient take), the conditions under which one or more reminders are provided to the patient, the content of the one or more reminders, the conditions under which a second notification is provided to another patient, the conditions under which an emergency physician is contacted, the rate at which blood pressure measurements (any optionally one or more other measurements) are taken.

FIG. 4 shows schematically by way of example a first computer system (1) connected to one or more sensors (10) and to a second computer system (1′). The first computer system (1) can be used by a patient; the second computer system (1′) can be used by another person, e.g., a physician.

The first computer system (1) and the second computer system (1′) can be connected via one or more networks, such as a mobile phone network and/or the internet.

The first and/or the second computer system may be designed as a smartwatch, a mobile phone (smartphone), a tablet computer, a desktop computer, or a computer tower.

The one or more sensors (10) can be connected to the first computer system (1) via cable or via a short-range communications technology such as NFC, RFID, Bluetooth, Bluetooth LE, ZigBee, infrared and/or the like. The one or more sensors (10) can also be part of the first computer system (1).

The one or more sensors (10) are configured to continuously measure the blood pressure of a patient (and optionally further patient data). The measured blood pressure data (and optionally further patient data) are transmitted to the first computer system (1) and/or the second computer system (2) and analyzed. If such analysis produces evidence of an emerging or existing hypertensive crisis, a first notification is provided to the patient via the first computer system. The notification comprises a request for the patient to perform an action. If the patient does not perform the action within a pre-defined time period, and/or or if other events described herein occur or do not occur, a second notification may be provided to the other person via the second computer system (1′).

FIG. 5 shows schematically by way of example a computer system (1) connected to one or more sensors (10) and to a medication dispensing device (20). The computer system (1) can be used by a patient.

The computer system may be designed as a smartwatch, a mobile phone (smartphone), a tablet computer, a desktop computer, or a computer tower.

The one or more sensors (10) can be connected to the computer system (1) via cable or via a short-range communications technology such as NFC, RFID, Bluetooth, Bluetooth LE, ZigBee, infrared and/or the like. The one or more sensors (10) can also be part of the computer system (1).

The medication dispensing device (20) can be connected to the computer system (1) via cable or via a short-range communications technology such as NFC, RFID, Bluetooth, Bluetooth LE, ZigBee, infrared and/or the like. The medication dispensing device (20) can also be part of the computer system (1).

The one or more sensors (10) are configured to continuously measure the blood pressure of a patient (and optionally further patient data). The measured blood pressure data (and optionally further patient data) are transmitted to the computer system (1) and analyzed. If such analysis produces evidence of an emerging or existing hypertensive crisis, and/or if blood pressure values do not fall below a pre-defined limit within a pre-defined period of time, the medication dispensing device (20) may be triggered by the computer system (1) to administer to the patient a pre-defined amount of a pre-defined medication to lower the blood pressure.

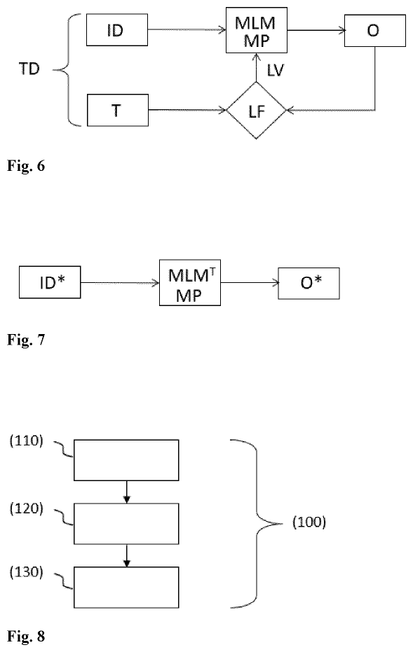

FIG. 6 shows schematically by way of example the process of training a machine learning model. The machine learning model MLM is trained on the basis of training data. The training data comprise a multitude of data sets, each data set comprising input data and target data. In the example shown in FIG. 6, only one training data set TD comprising input data ID and target data T is shown. The input data ID is inputted into the machine learning model MLM. The machine learning model is configured to generate, at least partially on the basis of the input data ID and model parameters MP, an output O. The output O is compared with the target T. This is done by using a loss function LF, the loss function quantifying the deviations between the output O and the target T. For each pair of an output O and the respective target T, a loss value is computed. During training the model parameters are modified in a way that reduces the loss values to a defined minimum. The aim of the training is to let the machine learning model generate for each input data an output which comes as close to the corresponding target as possible. Once the defined minimum is reached, the (now fully trained) machine learning model can be used to predict an output for new input data (input data which have not been used during training and for which the target is usually not (yet) known).

FIG. 7 shows schematically by way of example how a trained machine learning model can be used for making predictions. The trained machine learning model MLM^{T} can be the machine learning model described with reference to FIG. 6. New input data ID* are inputted into the trained machine learning model MLM^{T}. The trained machine learning model MLM^{T} is configured and trained to generate, at least partially on the basis of the new input data ID* and the model parameters MD, an output O*.

The (new) input data comprise patient data, in particular, blood pressure data over time. The output data depends on how the machine learning model is configured.

In one embodiment, the machine learning model is configured and trained to determine a probability value based on the input data, the probability value indicating how likely it is that the patient will experience a hypertensive crisis in an imminent time period (for example, in the next hour, or the next half hour). If, for example, the probability value is above a pre-defined threshold, for example higher than 50% or 60% or 70% or any other value, then the computer system can be configured to provide a notification to the patient and/or another person.

In another embodiment, the model is configured and trained to predict, on the basis of the input data, the patient's blood pressure in an imminent time period (for example, in the next hour, or the next half hour). If, for example, the predicted pressure is above a pre-defined threshold, then the computer system can be configured to provide a notification to the patient and/or another person.

FIG. 8 shows schematically in the form of a flowchart one embodiment of the computer-implemented method of the present disclosure.

The method (100) comprises the steps:
(110) receiving patient data, the patient data comprising data about a patient's blood pressure over time,
(120) analyzing the patient data and identifying one or more signs of a hypertensive crisis in the patient data,
(130) providing a notification to the patient, the notification comprising information about the identified signs of the hypertensive crisis and a request for the patient to perform an action.

1. A computer-implemented method, the method comprising:
receiving patient data, the patient data comprising data about a patient's blood pressure over time,
analyzing the patient data and identifying one or more signs of a hypertensive crisis in the patient data, and
providing a notification to the patient, the notification comprising information about the identified one or more signs of the hypertensive crisis and a request for the patient to perform an action.

2. The method according to claim 1, wherein the one or more signs of the hypertensive crisis comprise one or more measured and/or predicted blood pressure values exceeding one or more pre-defined thresholds.

3. The method according to claim 1, wherein the one or more signs of the hypertensive crisis comprise a probability value exceeding pre-defined threshold, the probability value indicating the probability of occurrence of the hypertensive crisis within a pre-defined imminent time period.

4. The method according to claim 2, wherein the one or more pre-defined thresholds were determined individually for the patient.

5. The method according to claim 1, wherein the rate at which blood pressure measurements are taken is automatically adjusted over time to the degree of change of the patient's blood pressure.

6. The method according to claim 1, wherein a reminder is provided to the patient and/or a second notification is provided to another person, if the patient does not perform the requested action within a pre-defined time period.

7. The method according to claim 2, wherein a reminder is provided to the patient and/or a second notification is provided to another person, if the patient's blood pressure exceeds the one or more pre-defined thresholds.

8. The method according to claim 2, wherein a second notification is provided to another person, if the patient's blood pressure does not fall below the one or more pre-defined thresholds within a pre-defined time period.

9. The method according to claim 8, wherein the second notification comprises one or more of the following: actual blood pressure of the patient, the patient's blood pressure history, geo-coordinates of the patient, information about actions performed and/or not performed by the patient.

10. The method according to claim 2, wherein a medication dispensing device is triggered to automatically administer a medication, if the patient's blood pressure exceeds the one or more pre-defined thresholds, and/or if the patient's blood pressure does not fall below the one or more pre-defined thresholds within the pre-defined time period, and/or if the patient does not perform the requested action.

11. The method according to claim 1, further comprising
inputting the patient data into a trained machine learning model,
receiving from the trained machine learning model
i) a predicted blood pressure value, the predicted blood pressure value indicating the expected patient's blood pressure that will be reached for a defined period in the immediate future, or
ii) a probability value, the probability value indicating the probability of occurrence of the hypertensive crisis within a pre-defined imminent time period,
comparing the predicted blood pressure value/the probability value with a pre-defined threshold, and
in case the predicted blood pressure value/the probability value exceeds the pre-defined threshold: providing the first notification to the patient and/or providing a reminder to the patient and/or providing a second notification to another person.

12. A computer system comprising:
a processor; and
a memory storing an application program configured to perform, when executed by the processor, an operation, the operation comprising:
receiving patient data, the patient data comprising data about a patient's blood pressure over time,
analyzing the patient data and identifying one or more signs of a hypertensive crisis in the patient data, and
providing a notification to the patient, the notification comprising information about the identified one or more signs of the hypertensive crisis and a request for the patient to perform an action.

13. A non-transitory computer readable medium having stored thereon software instructions that, when executed by a processor of a computer system, cause the computer system to execute the following steps:
receiving patient data, the patient data comprising data about a patient's blood pressure over time,
analyzing the patient data and identifying one or more signs of a hypertensive crisis in the patient data, and
providing a notification to the patient, the notification comprising information about the identified one or more signs of the hypertensive crisis and a request for the patient to perform an action.

14. The system according to claim 12, wherein the one or more signs of the hypertensive crisis comprise one or more measured and/or predicted blood pressure values exceeding one or more pre-defined thresholds.

15. The system according to claim 12, wherein the one or more signs of the hypertensive crisis comprise a probability value exceeding pre-defined thresholds, the probability value indicating the probability of occurrence of the hypertensive crisis within a pre-defined imminent time period.

16. The system according to claim 14, wherein the one or more pre-defined thresholds were determined individually for the patient.

17. The system according to claim 12, wherein the rate at which blood pressure measurements are taken is automatically adjusted over time to the degree of change of the patient's blood pressure.

18. The system according to claim 12, wherein a reminder is provided to the patient and/or a second notification is provided to another person, if the patient does not perform the requested action within a pre-defined time period.

19. The system according to claim 14, wherein a reminder is provided to the patient and/or a second notification is provided to another person, if the patient's blood pressure exceeds the one or more pre-defined thresholds.

20. The system according to claim 14, wherein a second notification is provided to another person, if the patient's blood pressure does not fall below the one or more pre-defined thresholds within a pre-defined time period.
